(19) United States
(12) Patent Application Publication
(10) Pub. No.: US 2018/0055680 A1
(43) Pub. Date: Mar. 1, 2018

(54) LIFE APPLICATION PATCH

(71) Applicant: Kathleen Susan Canavan, Bohemia, NY (US)

(72) Inventor: Kathleen Susan Canavan, Bohemia, NY (US)

(21) Appl. No.: 15/692,901

(22) Filed: Aug. 31, 2017

Related U.S. Application Data
(60) Provisional application No. 62/495,027, filed on Aug. 31, 2016.

(51) Int. Cl. A61F 5/44; A61F 5/443; A61F 5/445

(52) U.S. Cl. CPC A61F 5/4404; A61F 5/445; A61F 5/443; A61F 5/4401

(57) ABSTRACT

A stoma dressing for use by ostomy patients comprising an underlying fluid impermeable layer having an adhesive edge; a highly absorbent core fixedly superimposed on the underlying fluid impermeable layer and totally encircled by the adhesive edge; a groove running the entire outer perimeter of the middle highly absorbent core; and a bead of skin healing/skin protecting cream or ointment filling the groove. In use, the stoma dressing is placed over the stoma in a manner that the top of the highly absorbent core and the adhesive edge of the impermeable layer face the skin and stoma of the patient. When lightly pressed against the skin such that the adhesive edge adheres to the patient's skin, the bead of cream or ointment in the groove spreads on the patient's skin around the outer perimeter of the middle absorbent core, to create a skin area that is protected from the stoma effluents without interfering with the adhesive properties of the adhesive edge of the dressing.

### CROSS-REFERENCE TO RELATED APPLICATIONS

This application claims priority from U.S. Provisional Application Ser. No. 62/495,027 filed on Aug. 31, 2016, and incorporated by reference in its entirety, as if more fully set forth herein.

### BACKGROUND OF THE INVENTION

### Field of Invention

The present invention relates to ostomy bandages or dressings, for use by ostomy patients, i.e., patients who have undergone the surgical construction of an artificial excretory opening or stoma. More particularly, the present invention relates to an ostomy bandage comprising a highly absorbent core mounted on a moisture barrier layer, a groove on the outer perimeter of the core, and a hydrophobic composition, such as an ointment or a cream contained within the groove such that the ostomy bandage is highly absorbent, easily removed to provide access to the stoma and thereafter easily reattached to the skin surrounding the stoma, if necessary. It is highly effective in preventing the soiling of the patients' clothing while at the same time promoting the healing of and the protecting of the skin around the stoma, preventing injury and infection thereto, and prohibiting the spreading of bodily fluids and waste beyond the outer perimeter of the core.

### Prior Art

The terms “ostomy” and “stoma” are general descriptive terms that are often used interchangeably, though they have different meanings. An ostomy refers to the process of surgically creating an opening in the body for the discharge of body wastes. A stoma is the actual end of the ureter, or small, or large bowel that can be seen protruding through the abdominal wall, after an ostomy is completed. The most common specific types of ostomies are colostomies, ileostomies, and urostomies.

As of October 2016, more than 750,000 Americans live with an ostomy, and over 100,000 Americans undergo an ostomy every year. Whether it is because of cancer; or an inflammatory bowel disease like Crohn's disease, or ulcerative colitis, diverticulitis, or incontinence, an ostomy can give people with debilitating illnesses a new lease on life-one with fewer hospitalizations and less debilitating pain.

In some ostomies, an external pouch is attached to the stoma to provide a reservoir for the collection of the waste. In other ostomies, such as a urostomy where, for example, an internal continent Kock or Indiana pouch is installed with an abdominal stoma, no external pouch is necessary. If a pouch is used to accumulate the stoma secretions, then the use of a skin barrier to protect the peri-stomal skin and promote healing around the stoma is highly recommended. In the case of a non-pouch urostomy, the skin barrier is not necessarily recommended. A skin barrier does not stop the leaking of urine through the stoma once the internal pouch is filled. Instead the internal continent pouch requires convenient catheterization through the abdominal stoma. More specifically the continent pouch is emptied by inserting a flexible silicone catheter with a coude'tip (elbow or angled tip) into the stoma four to eight times a day. Following catheterization or in a regulated colostomate, a small moisture proof pad needs to be worn over the stoma to absorb normal stomal secretions.

Ostomy patients unfortunately don't have many choices when it comes to small moisture proof pads for use over the stoma to absorb normal stomal secretions, such as leaking urine, and whatever choices they do have are poor ones. For example, BANDAID® brand bandages don't work because they are just not absorbent enough. Mucus leaks through to soil the ostomy patient's clothing. http://www.j-pouch.org/topic/stoma-covering. Urine leaks through too. When the pouch or bladder reaches capacity, the urine spurts out of the stoma uncontrollably.

3M currently manufactures and sells a colostomy dressing under the mark NEXCARE™ STOMASEAL™. 3M markets it as “a time-saving application for the regulated colostomate, featuring pad & adhesive in an easy-to-apply dressing that is comfortable to wear.” The adhesive is “strong to stick tightly and securely, but removes easily.” “The pad absorbs 6 times its own weight in bodily fluids.” It “protects clothing because the tape resists fluid penetration.” http://www.nexcare.com/3M/en_US/nexcare/products/catalog/˜/Nexcare-Stomaseal-Colostomy-Dressing?N=4326+3294529207+3294631188&rt=rud. However, while the NEXCARE™ STOMASEAL™ Colostomy Dressings work a bit better than the Bandaid® bandages, they do not work well with urostomy patients. Despite the fact that they absorb 6 times their own weight in bodily fluids, they are still just not absorbent enough. In the absence of a timely catheterization, urine leaks through to soil the ostomy patient's clothing, and after repeated leaks can injure or lead to the breakdown of the ostomy patients' peri-stomal skin.

MESTOPORE S™ dressings are being marketed as self-adhesive dressings with the Safetac™ soft silicone technology for continent stomas, capable of being removed with minimal trauma and pain and provided with a highly absorbent pad. https://marchesemedicalsupplies.ca/mestopore-s-dressing-10 cm-x-10 cm Safetac® is patented soft silicone adhesive technology. Dressings with Safetac® technology are atraumatic both during wear and upon removal. These dressings minimize trauma to the wound and the surrounding skin, by preventing skin stripping upon removal of the dressings.

While MESTOPORE S dressings claim to have highly absorbent pads, it only takes an hour or two for the pads to become soaked. As a result, even MESTOPORE S pads are not absorbent enough. In the absence of a timely catheterization of the internal pouch created by a urostomy, urine leaks through to soil the ostomy patient's clothing, and after repeated leaks can injure or lead to the breakdown of the ostomy patients' peri-stomal skin.

As a result, ostomy patients are constrained to resort to all sorts of other items to absorb their stomal secretions, including leaking urine. Some patients use nursing pads held in place with tape. Other patients use cotton balls covered with makeup remover pads, secured with tape right over their stoma. Still others, use sanitary, or menstrual napkins or pads such as KOTEX® or ALWAYS®, also held in place with tape. While still others, use tissue paper, napkins, or toilette paper. As one can imagine, these are poor solutions to the ostomy patients' need for small moisture proof pads for use over the stoma, to absorb normal stomal secretions and urine, and prevent the soiling of their clothing.

The bigger problem with the foregoing solutions is the skin irritation and ultimately skin injury that they can cause. The stomal secretions, absorbed by the bandages and coverings discussed above, will diffuse throughout the entire dressing, accumulate under the adhesive tape, and irritate and breakdown the pen-stomal skin. For example, ileostomy output is full of digestive enzymes, which if they leak and come in contact with the peristomal skin, can lead to skin breakdown. Likewise, urine spurting out of the stoma uncontrollably, when the internal urostomy pouch or bladder reaches capacity, can and will lead to skin break down. The urine accumulates under the adhesive perimeter of the dressings to irritate the peristomal skin to which the dressings are attached.

According to www.ostomy.org the best skin protection is a well-fitted and comfortable pouching system, if applicable. In addition, there are a number of basic ostomy skin care products that are available to help skin that tears easily. Such products include skin sealants, adhesive removers, skin bather pastes, ostomy adhesives, and skin barrier powders. On the other hand, the use of skin healing oil based products such as oil based soaps, white, petrolatum, vitamin A&D ointment, zinc oxide, lanolin, creams, lotions and other similar products are contra-indicated because they are greasy and oily. They interfere with the adhesion of ostomy devices or the dressings set forth herein above and exacerbate the leakage problems and therefore the skin irritation associated therewith. Consequently, some patients with flesh to skin stomas that leak urine from the abdomen require Botox injections to paralyze the nerves in the bladder to prevent leakage; a very extreme solution to the problem of stoma mucus and urine leakage.

Quality of life is paramount for ostomy patients. Bandages or dressings of the type described above and the problems they can cause will and do affect the lives of ostomy patients. Leaks lead to frequent changes in clothes and frequent emptying. Not only are frequent leaks and emptying inconvenient, but they make patients feel self-conscious about their appearance, their body odor, their overall physical attractiveness, sexual function, and can potentially get them down and depressed causing them not to want to go out or see people, leading to social isolation.

Accordingly, there is a real need for the development of moisture proof bandages or dressings that can be worn over the stoma for long periods of time, capable of absorbing normal stomal secretions and urine leaks. There is a need for dressings that are highly absorbent and effective in preventing the soiling of the patients' clothing, while at the same time promoting the healing of and the protecting of the skin around the stoma, preventing injury and infection thereto, and prohibiting the spreading of bodily fluids and waste beyond the perimeter of the bandages or dressings. Finally, there is a real need for ostomy bandages or dressings capable of helping patients recapture their quality of life and self-confidence, and restoring to them their mental health and well being.

### SUMMARY OF THE INVENTION

Accordingly, it is an object of the present invention to provide stoma bandages or dressings capable of absorbing bodily fluids for long periods of time by means of a highly absorptive central core material.

It is another object of the present invention to provide stoma bandages or dressings capable of preventing injury to, or the breakdown of the ostomy patients' peri-stomal skin.

It is still another object of the present invention to provide stoma bandages or dressings capable of preventing the migration of stoma effluents such as mucus or urine to, and their interference with, the adhesive edge of said bandages or dressings, thereby preventing the dressings from falling off the stoma on their own.

It is yet object of the present invention to provide stoma dressings or bandages that provide for and promote healthy peri-stomal skin.

It is a further object of the present invention to provide stoma dressings or bandages that allow for the use of skin protecting creams or ointments, without interfering with the adhesive capabilities of said dressings to the skin, surrounding a stoma.

It is an object of the present invention to provide stoma bandages or dressings with a breathable adhesive band all around the dressings, capable of securing the dressings to the particular area of skin surrounding the stoma.

It is yet another object of the present invention to provide stoma dressings that are highly absorbent and effective in preventing the soiling of the patients' clothing while at the same time promoting the healing of and the protecting of the skin around the stoma, preventing injury and infection thereto, and prohibiting the spreading of bodily fluids and waste beyond the perimeter of the bandages or dressings.

It is still another object of the present invention to provide stoma bandages or dressings capable of helping patients recapture their quality of life and self-confidence, and restoring their mental health and well being.

In accordance with the present invention there is provided a stoma bandage or dressing comprising an underlying fluid impermeable layer having an adhesive edge; a middle, thick, highly absorbent core superimposed on the underlying fluid impermeable layer and totally encircled by the adhesive edge; a groove running the entire outer perimeter of the middle highly absorbent core; and a bead of skin healing/skin protecting cream or ointment filling the groove. In use, the inventive stoma bandage or dressing is placed over the stoma in, a manner that the top of the middle highly absorbent core and the adhesive edge of the impermeable layer face the skin and stoma of the patient. When lightly pressed against the skin such that the adhesive edge adheres to the patient's skin, the bead of cream or ointment in the groove spreads on the patient's skin around the outer perimeter of the middle absorbent core, to create a skin area that is protected from the stoma effluents, without interfering with the adhesive properties of the outer adhesive edge of the dressing.

These and other objects, advantages, features, and characteristics of the invention will be apparent from the following description of the preferred embodiments, considered along with the accompanying drawings.

### BRIEF DESCRIPTION OF THE DRAWINGS

It is believed that the present invention will be better understood from the following detailed description taken in conjunction with the accompanying drawings in which the numerals represent identical elements and wherein:

FIG. 1 is a three dimensional perspective of one embodiment of the inventive stoma dressing.

FIG. 1A is a top plan view of the inventive stoma dressing of FIG. 1.

FIG. 1B is a cross section side view of the inventive stoma bandage taken along the line A-A, in FIG. 1A.

FIG. 2 is a three dimensional perspective of another embodiment of the inventive stoma dressing.

FIG. 2A is a top plan view of the inventive stoma dressing of FIG. 2.

FIG. 3 is a three dimensional perspective of still another embodiment of the inventive stoma dressing.

FIG. 3A is a top plan view of the inventive stoma dressing of FIG. 3.

FIG. 3B is a cross section side view of the inventive stoma bandage taken along the line C-C, in FIG. 3A.

FIG. 4 is a three dimensional perspective of yet another embodiment of the inventive stoma dressing.

FIG. 4A is a top plan view of the inventive stoma dressing of FIG. 4.

FIG. 5 is a three dimensional perspective of another embodiment of the inventive stoma dressing.

FIG. 5A is a top plan view of the inventive stoma dressing of FIG. 5.

FIG. 5B is a cross section side view of the inventive stoma bandage taken along the line B-B, in FIG. 5A.

### LIST OF ELEMENTS AND THEIR RESPECTIVE IDENTIFYING NUMERALS

NO ELEMENT
10 inventive stoma dressing
20 fluid impermeable layer
22 adhesive edge
30 absorbent core
40 groove
42 bead of cream or ointment

### DETAILED DESCRIPTION OF THE PREFERRED EMBODIMENT

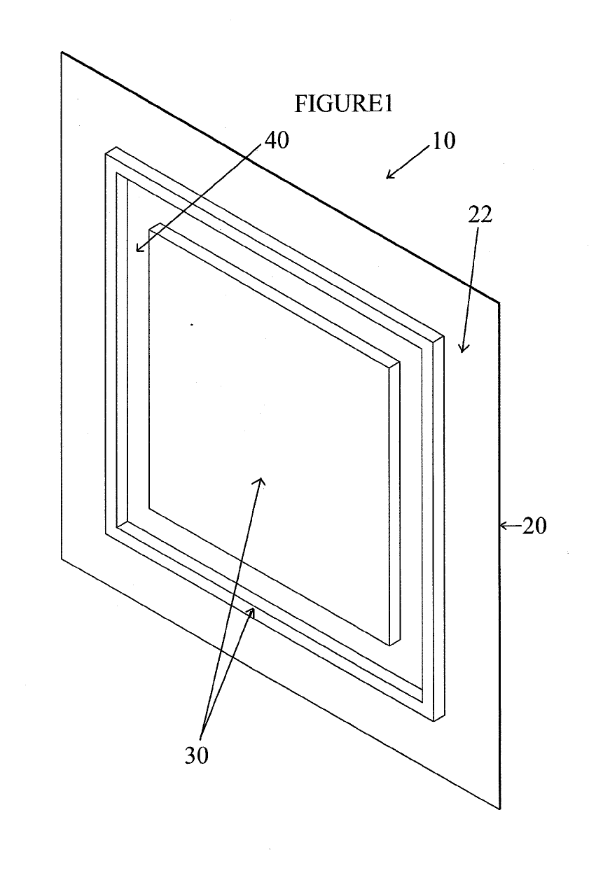

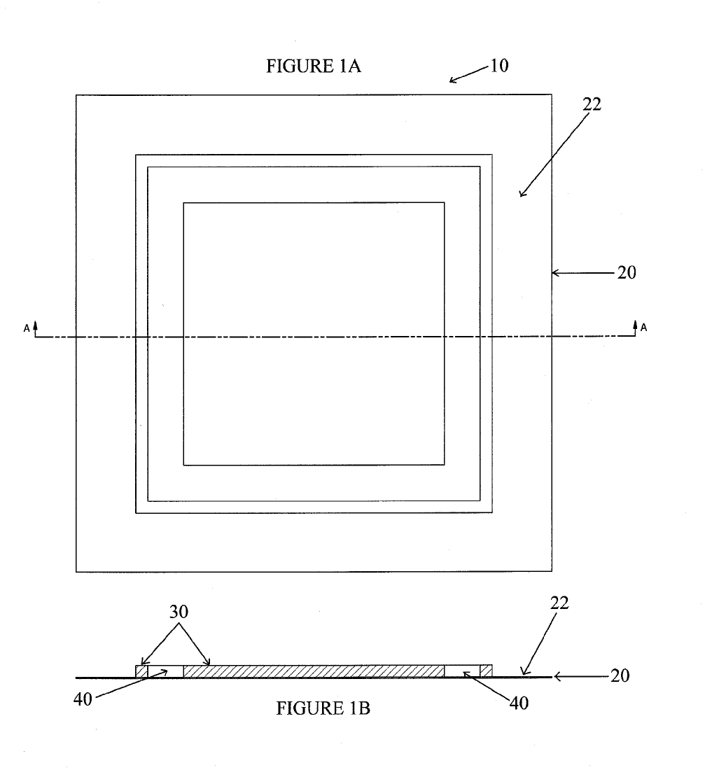

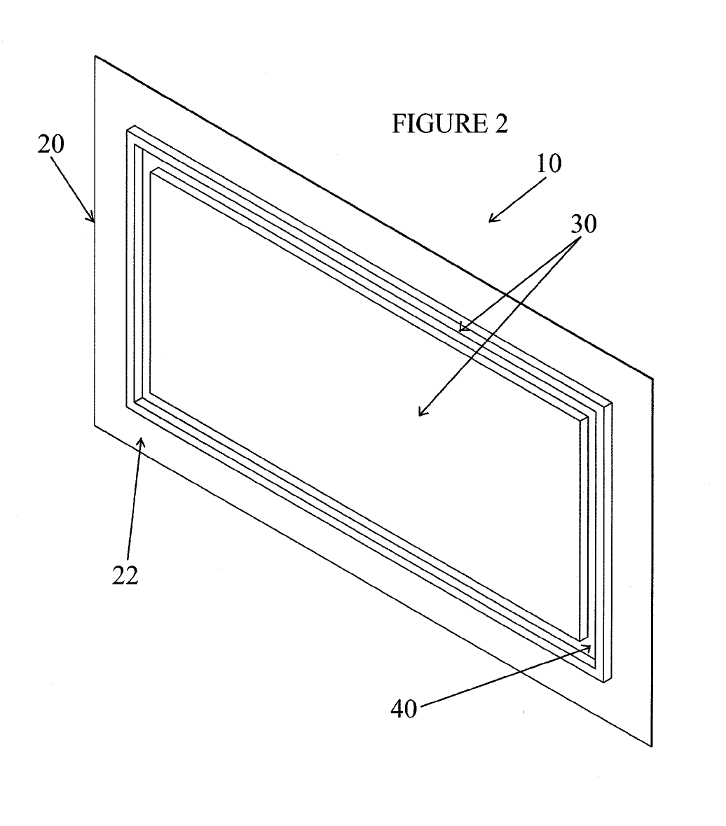

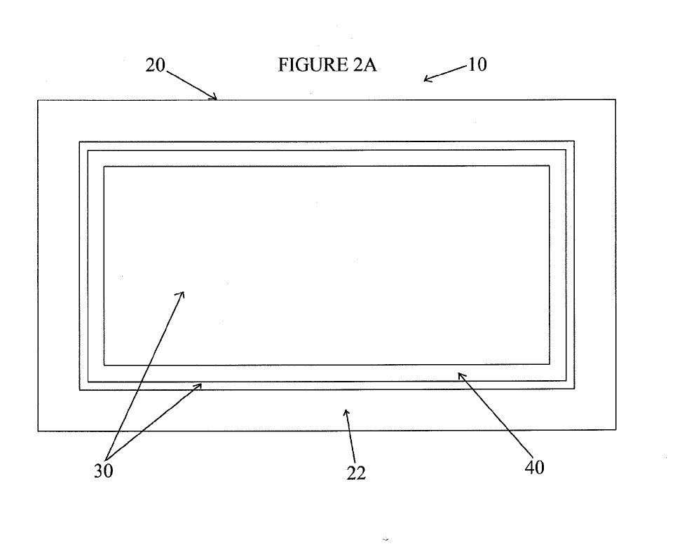

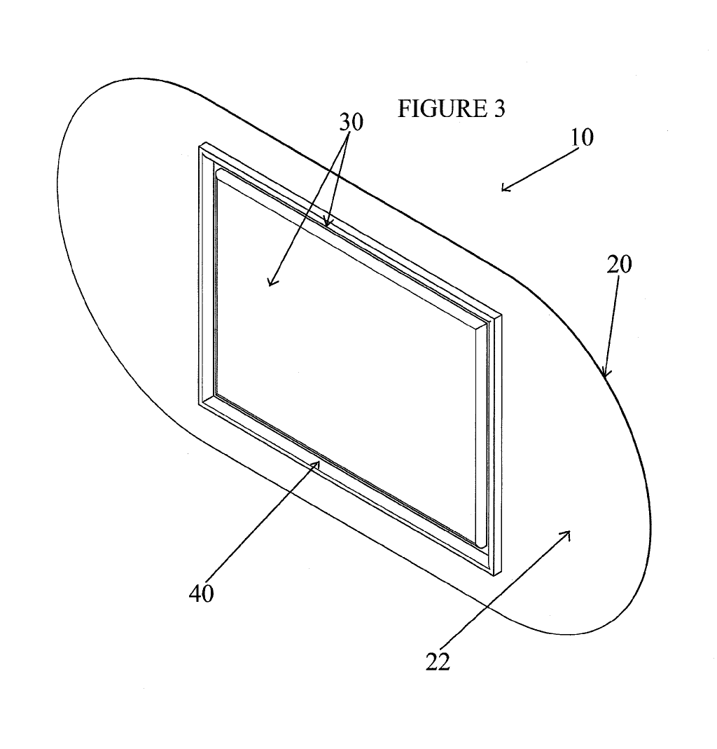

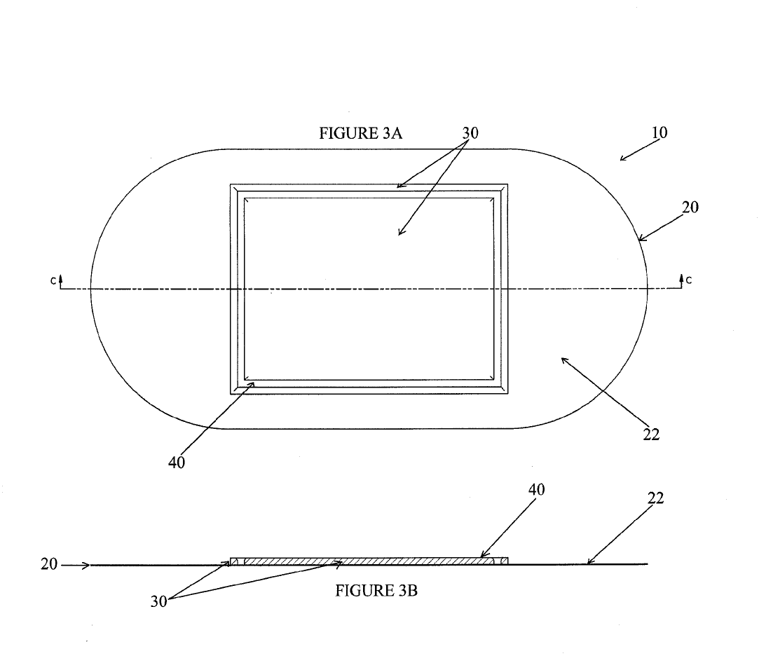

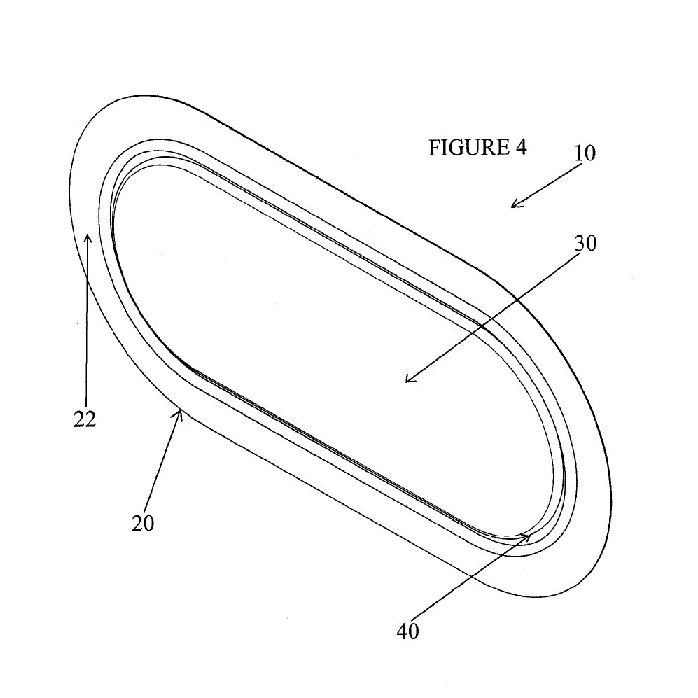

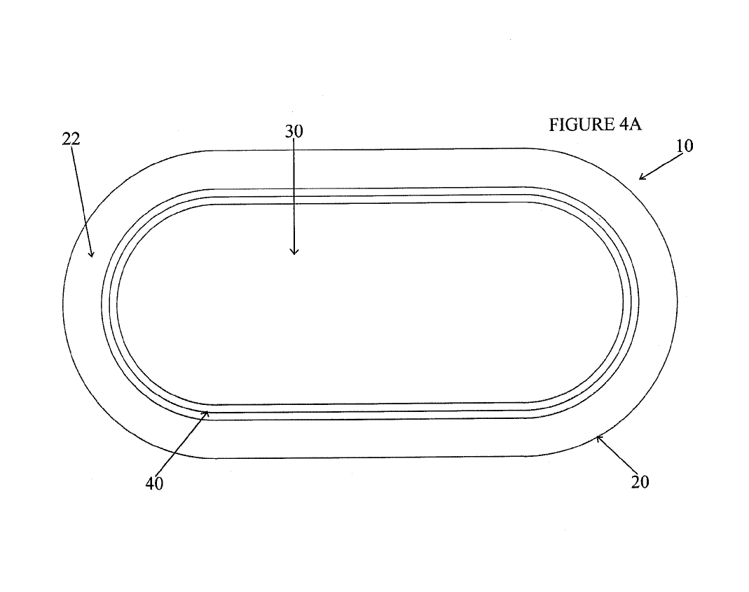

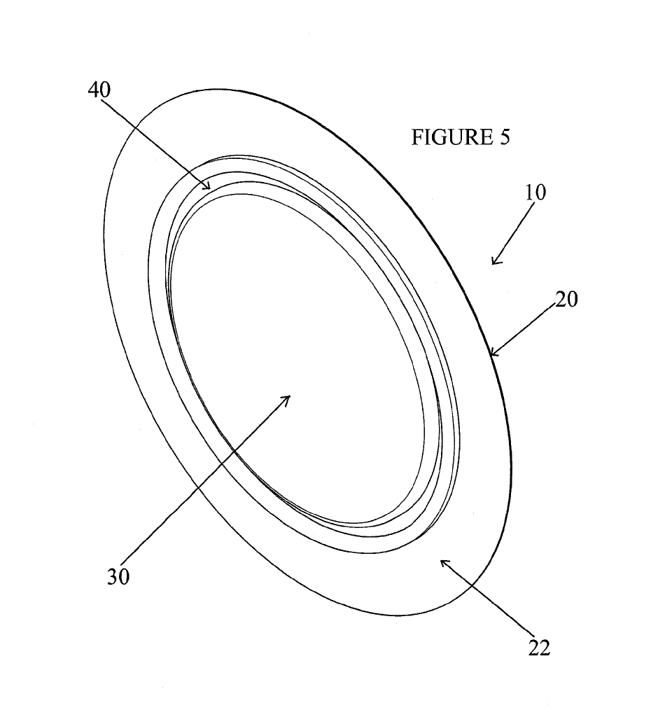

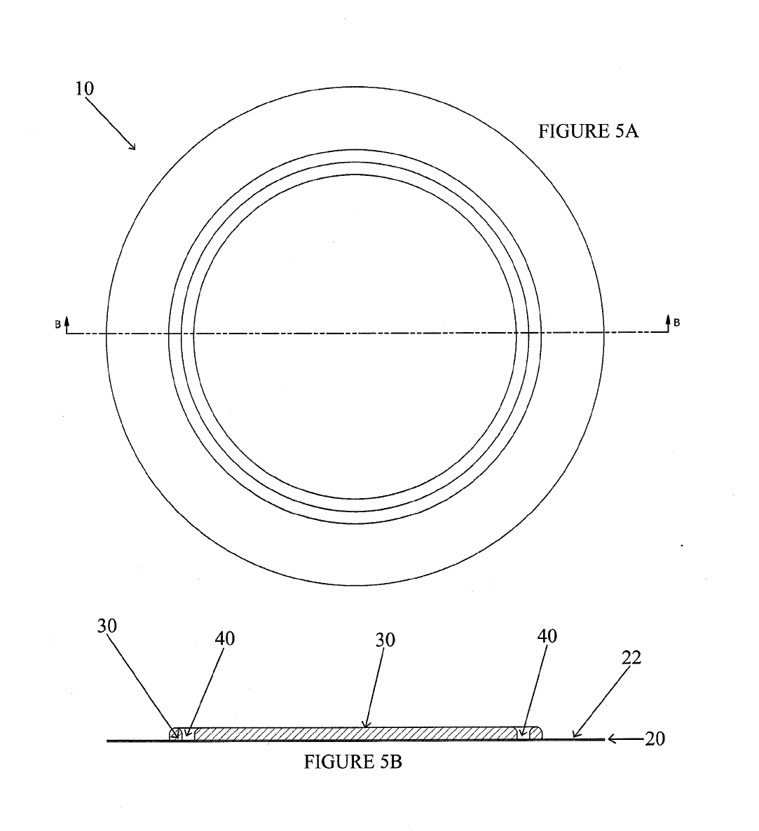

Referring more specifically to FIGS. 1-5, they generally depict different embodiments of the inventive stoma dressing in accordance with the present invention at 10, which can be used by ostomy patients. It comprises an underlying fluid impermeable layer 20 having an adhesive edge 22; a middle, thick, highly absorbent core 30, fixedly superimposed on the underlying fluid impermeable layer 20, and totally encircled by the adhesive edge 22; a groove 40 running the entire outer perimeter of the middle highly absorbent core 30; and a bead of skin healing/skin protecting cream or ointment 42 filling the groove 40. Optionally, an additional top layer or surface layer 32 is superimposed on the middle, thick, highly absorbent core 30. In use, the optional, additional surface layer 32 sits right against the skin. It acts as a moisture barrier between the skin and the highly absorbent core 30 to keep the skin dry and comfortable.

The underlying fluid impermeable layer 20 acts as a support for the highly absorbent core 30. In use, it sits right against the patient's undergarments or clothes. It acts as a liquid impermeable, moisture penetrable barrier between the absorbent core 30 and the garments of the ostomy patient, keeping the ostomy patient dry and comfortable, while simultaneously allowing the patient's skin to breath, and preventing the growth of bacteria. As a result it can be made, or thermally formed, using any process currently disclosed in the prior art, or to be disclose in the future, of any material, or combination of materials, and have any structure that will produce a fluid impermeable but moisture penetable thin layer or sheet, that can act as a moisture barrier between the absorptive core 20 and the ostomy patients' garments, while at the same time being permeable to vapor or moisture. By way of example, some of the materials that could be used to form the impermeable layer 20, can include but are not limited to, vinyl, polyethylene, polyvinylchloride, mylar sheeting, or any combination of cellulose and manmade materials or fibers. Optionally, it may also be made of silicon using SAFETAC soft silicone technology.

The underlying fluid impermeable layer 20 has an outer perimeter provided with adhesive to form an adhesive edge 22. The adhesive must be strong enough to allow for the tight, secure but removable adhesion of inventive stoma bandage 10 to the patient's skin, on the area surrounding the patient's stoma. The adhesive could be any adhesive selected from the group of adhesives used in the prior art in connection with medical tape, wound dressings and such currently available or to become available, which is incorporated in its totality, by reference, as if more fully set forth herein. Alternatively the adhesive edge could be formed using SAFETAC® soft silicone technology, because it will permit for the gentle adhesion to the surrounding skin, minimize trauma and pain on removal and protect against the risk of maceration. Optionally, the adhesive edge 22 could be covered with a protective liner which can be removed when the stoma dressing 10 is prepared for application over the patient's stoma.

As was set forth above and as shown in FIGS. 1-5, the absorbent core 30 is fixedly mounted on the fluid impermeable layer 20. It is made of a highly absorbent material capable of absorbing human exudates such as blood, puss, discharge and urine. Such highly absorbent material can be selected from the group of highly absorbent materials currently available, or to become available, in the prior art for use in sanitary and incontinence pads, and incontinent undergarment technology, including but not limited to polyester, cotton fibers, rayon fibers, gel forming chemicals, silicon and so on. The preferred material will have integrity when wet. This highly absorptive material must wisk bodily fluid away from the skin preventing moisture and bacteria from maintaining contact with the patient's skin. Optionally it could be provided with odor neutralizing perfumes or technology currently available, or possible available in the future, which is hereby incorporated in its totality as if more fully set forth herein.

The absorbent core 30 rises above the underlying fluid impermeable layer 20 and is of sufficient dimensions and thickness such that it allows for the absorbance of stoma exudates for more than just a few hours. The outer perimeter of the absorbent core 30, in turn is provided with a groove 40 using any of the prior art groove forming prior art technology currently available, or to become available in the future, which is incorporated in its totality, as if more fully set forth herein.

The groove 40 has an inner wall and an outer wall. It runs the entire length of the absorbent core 30's outer perimeter. It functions as a receptacle for a bead of skin protectant/skin healing cream or ointment 42, its inner and outer walls containing the bead of cream or ointment therein, away from the adhesive edge 22 of the fluid impermeable layer 20, until the inventive stoma bandage 10 is used. In use, while the adhesive edge 22 of the stoma dressing 10 is gently pressed and adhered on the skin surrounding the patient's stoma, the bead of cream or ointment in the groove spreads on the patient's skin around the outer perimeter of the middle absorbent core, to create a skin area that is protected from the stoma effluents without interfering with the adhesive properties of the outer adhesive edge of the dressing.

As was discussed in the prior art above, the use of any oil containing, or greasy feeling cream or ointment is counter-indicated in ostomy type devices because it interferes with the proper adhesion of the ostomy devices to the area of the skin surrounding the patient's stoma. In the stoma dressing 10 of the present invention, the location of the groove 40 on the outer perimeter of the absorbent core 30, the placement of the bead of cream or ointment within the groove, the containment of the bead 42 within the walls of the groove 40, and the height of the groove's walls within the thick absorbent core 30, allows the cream or ointment to spread only after the adhesive edge is pressed onto the skin, without significant interference with the adhesion capabilities of the adhesive edge. Further the spread of cream or ointment forms a hydrophobic skin barrier, which completely encircles the absorbent core 30 and the stoma it covers during use. This hydrophobic skin barrier prevents the stoma exudates from migrating to and under the adhesive edge 22, thereby preventing the stoma dressing 10 of the present invention from falling off.

The creams of ointments that can be used to fill the groove 40 are chosen from the group of creams and ointments currently available, or might become available in the future for the treatment of skin breakdowns, irritations, rashes and injuries, and the soothing thereof. Such creams and ointments can include but are not limited to dimenthicone creams and ointments; lanolin based creams and ointments; aloe vera creams and ointments, and zinc oxide creams and ointments.

There is no question that the stoma dressing 10 of the present invention as described herein above, accomplishes all of its objectives. It is highly absorbent and effective in preventing the soiling of the patients' clothing. In practice, the stoma dressing 10 of the present invention was able to absorb urine for as much as 4-5 hours after catheterization of the internal urostomy pouch, allowing its user to resume all of her regular activities including taking long trips, without any accidents and without necessitating the change of clothes. Its skin barrier created after its application over the stoma promotes the healing of and protects the skin around the stoma, thereby preventing injury and infection thereto. It effectively prevents the stoma exudates from migrating to and under the adhesive edges of the stoma dressing 10, thereby preventing the stoma dressing 10 of the present invention from falling off. Finally, as a result of all of the foregoing, the stoma dressing of the present invention helps patients recapture their quality of life and self-confidence, and restore their mental health and well being.

While particular embodiments of the invention have been illustrated and described in detail herein, they are provided by way of illustration only and should not be construed to limit the invention. Since certain changes may be made without departing from the scope of the present invention, it is intended that all matter contained in the above description, or shown in the accompanying drawings be interpreted as illustrative and not in a literal sense. Practitioners of the art will realize that the sequence of steps and the embodiments depicted in the figures can be altered without departing from the scope of the present invention and that the illustrations contained herein are singular examples of a multitude of possible depictions of the present invention.

Accordingly, I claim:

1. A Stoma dressing for use by ostomy patients comprising an underlying fluid impermeable layer having an adhesive edge; a highly absorbent core fixedly superimposed on said underlying fluid impermeable layer, and totally encircled by said adhesive edge; a groove running the entire outer perimeter of said highly absorbent core and containing a bead of skin healing and skin protecting composition.

2. The Stoma dressing according to claim 1, wherein said skin healing and skin protecting composition is a skin cream.

3. The Stoma dressing according to claim 1, wherein said skin healing and skin protecting composition is an ointment.

4. The Stoma dressing according to claim 2, further comprising a moisture barrier layer fixedly superimposed on said highly absorbent core.

5. The Stoma dressing according claim 3, further comprising a moisture barrier layer fixedly superimposed on said highly absorbent core.

6. The process of an ostomy patient using a stoma dressing comprising the following steps:
a. Securing a stoma dressing which has an underlying fluid impermeable layer having an adhesive edge; a highly absorbent core fixedly superimposed on said underlying fluid impermeable layer, and totally encircled by said adhesive edge; and a groove running the entire outer perimeter of said highly absorbent core, said groove containing a bead of skin healing and skin protecting composition;
b. Placing the stoma dressing over the ostomy patient's stoma in a manner that the top of said highly absorbent core and said adhesive edge of said fluid impermeable layer face the skin and stoma of the patient;
c. lightly pressing against the patient's peri-stomal skin such that said adhesive edge adheres to the patient's skin, and the bead of said skin healing and skin protecting composition spreads on the patient's skin around the outer perimeter of said middle absorbent core, to create a skin area that is protected from the stoma effluents without interfering with the adhesive properties of said adhesive edge of said stoma dressing.
